(19) United States
(12) Patent Application Publication
(10) Pub. No.: US 2025/0067747 A1
(43) Pub. Date: Feb. 27, 2025

(54) METHOD FOR SCREENING TARGETS BY DIA-BASED QUANTITATIVE CHEMICAL PROTEOMICS

(71) Applicant: Nanjing ChomiX Biotech Co., Ltd., Nanjing (CN)

(72) Inventors: Chu Wang, Beijing (CN); Fan Yang, Beijing (CN); Nan Chen, Nanjing (CN)

(21) Appl. No.: 18/724,677

(22) PCT Filed: Dec. 29, 2022
(86) PCT No.: PCT/CN2022/143156

§ 371 Date: Jun. 27, 2024

(30) Foreign Application Priority Data
Dec. 31, 2021 (CN) 202111665395.3

(51) Int. Cl. G01N 33/68

(52) U.S. Cl. CPC G01N 33/6818; G01N 33/6851; G01N 2570/00

(57) ABSTRACT

A method for screening targets by data independent acquisition (DIA)-based quantitative chemical proteomics, comprising covalently modifying specific active amino acids in a proteome with an active molecular probe; and quantitatively analyzing sites covalently-modified by the probe through DIA-based quantitative omics method, to obtain candidate targets. According to the method for screening targets by DIA-based quantitative chemical proteomics, DIA-based quantitative omics technique is applied into activity-based protein profiling (ABPP) to form DIA-ABPP, so that high coverage, high reproducibility and high precision target screening may be achieved, and corresponding technical support is provided for subsequent drug development.

The present invention claims priority to the filing date of the prior application filed with the China National Intellectual Property Administration on Dec. 31, 2021, with the patent application Ser. No. 20/211,1665395.3 and the invention title “METHOD FOR SCREENING TARGETS BY DIA-BASED QUANTITATIVE CHEMICAL PROTEOMICS”. The entire content of the above-mentioned prior application is incorporated herein by reference.

### TECHNICAL FIELD

The present application relates to the field of chemical proteomics, and specifically to a method for screening targets by DIA-based quantitative chemical proteomics.

### BACKGROUND

Chemical proteomics is one of the hot research directions of chemical biology. It combines active molecular probes with quantitative mass spectrometry methods to analyze proteins with specific functions or modifications in complex proteomes, as well as to discover target proteins that interact with a specific active compound molecule.

The most commonly used chemical proteomics strategy is activity-based protein profiling (ABPP). The principle of ABPP technique is to design an active molecular probe that can covalently bind to certain amino acids in active proteins, so that the corresponding active state of proteins with certain specific activities can be obtained from the proteome. The structure of small molecular probes generally has three parts: a reactive group, which can specifically identify certain amino acid sites, such as cysteine; a reporter tag; and an intermediate linker, usually a carbon chain or a polyethylene glycol chain, for connecting the reactive group and the reporter tag. There are two types of reporter tags, one is a fluorescent group for gel electrophoresis detection, and the other is biotin, which is affinity enriched by streptavidin resin and used in mass spectrometry to identify proteins bound by active molecular probes. At present, ABPP is considered a promising new generation of function-based proteomics technique and a bridge between proteomics and drug target research.

With the continuous development of research fields and quantitative omics methods, quantitation-based chemical proteomics has been increasingly applied to the analysis of unknown protein functions, the identification of active small molecule target proteins, the discovery of post-translational modification sites, and the screening of small molecule inhibitors. In order to further achieve accurate quantitative analysis of chemical proteomics, scientists have developed quantitative chemical proteomics technique based on isotope tags, including isoTOP (isotope-based tandem orthogonal proteolysis)-ABPP technique and rdTOP (tandem orthogonal proteolysis based on reductive dimethylation)-ABPP technique based on primary spectrum quantification, and MTRP (multiplexed thiol reactivity profiling) technique and SLC (streamlined cysteine)-ABPP technique based on secondary spectrum quantification.

In 2016, the Cravatt team at the Scripps Research Institute in the United States combined a quantitative chemical proteomics technique (isoTOP-ABPP) with drug discovery based on small electrophilic molecule fragments. Through the preparation of hundreds of samples, months of mass spectrometry acquisition, data integration and analysis, they found that more than 700 proteins that were previously considered to be unable to be functionally regulated by small molecules contained active cysteine sites that could be covalently-modified by specific electrophilic fragments, making these proteins to be regulatable targets again. However, this technique can only perform dual quantitative analysis, the strategy is relatively complicated and lengthy, the reproducibility is poor, the number of identifications is small, and the sensitivity is low; in 2020, the Gygi team at Harvard University developed an isobaric tag-based multiple quantitative chemical proteomics technique (SLC-ABPP) for covalent fragment screening. While it can achieve parallel quantitative analysis of multiple samples, the tag reagents are very expensive, the algorithm is complex, and the operability is poor. Therefore, it cannot meet the needs of large-scale high-throughput detection of chemical biologists. To this end, new optimized chemical proteomics techniques are urgently needed to meet the demands of large-scale high-throughput detection.

### SUMMARY

A method for screening targets by DIA-based quantitative chemical proteomics provided in the present application applies DIA-based quantitative omics technique into ABPP to form DIA-ABPP, which can achieve high coverage, high reproducibility and high precision target screening, and can meet the demands of large-scale high-throughput detection.

The present application provides a method for screening targets by DIA-based quantitative chemical proteomics, comprising:

covalently modifying specific active amino acids in a proteome with an active molecular probe; and

quantitatively analyzing sites covalently-modified by the probe through DIA-based quantitative omics technique to obtain candidate targets.

In one embodiment, the active amino acid is cysteine, lysine, tyrosine, methionine, glutamic acid or aspartic acid.

It should also be noted that the active amino acid involved in the present application may also be modification sites that can be enriched by other chemical probes, and such implementation still belongs to the protection scope of the present application, for example: guanidinium modification related to rheumatoid arthritis, see the literature for details: Ronak Tilvawala, Son Hong Nguyen, Aaron J. Maurais, Venkatesh V. Nemmara, Mitesh Nagar, Ari J. Salinger, Sunil Nagpal, Eranthie Weerapana, Paul R. Thompson. The Rheumatoid Arthritis-Associated Citrullinome [J]. Cell Chemical Biology, 2018, 25 (6).

In one embodiment, the active amino acid is cysteine.

An IA-alkyne probe, a broad-spectrum reactive probe, can be used for quantitative analysis of cysteine-reactivity.

In one embodiment, the correspondence between the active amino acid and the active molecular probe is: the cysteine being covalently-modified by an IA-alkyne probe, the lysine being covalently-modified by a tetrafluorophenylsulfonate-alkyne probe, the tyrosine being covalently-modified by a sulfonyltriazole-substituted alkyne probe, the methionine being covalently-modified by an oxazine-alkyne probe, and the glutamic acid and aspartic acid being covalently-modified by a 3-phenyl-2H-aziridine-alkyne probe.

In one embodiment, the correspondence between the active amino acid and the active molecular probe is: cysteine being covalently-modified by an IA-alkyne probe.

In one embodiment, the process of covalently modifying specific active amino acids in a proteome with an active molecular probe, wherein a cleavable tag is used, specifically comprising:

obtaining a proteome sample; treating the sample with the active molecular probe in the dark; subjecting the treated sample to a click chemistry reaction with the cleavable tag; after enrichment and digestion overnight, washing off non-specifically adsorbed peptides and urea, and finally performing cleavage using a cleavage method corresponding to the cleavable tag reagent.

In one embodiment, the process of covalently modifying specific active amino acids in a proteome with an active molecular probe, wherein an acid cleavable tag is used, specifically comprising:

obtaining a proteome sample; treating the sample with the IA-alkyne probe in the dark; subjecting the treated sample to a click chemistry reaction with the acid cleavable tag; after enrichment and trypsin digestion overnight, washing off non-specifically adsorbed peptides and urea, and finally performing cleavage using a cleavage method corresponding to the acid cleavable tag reagent.

In one embodiment, the process of quantitatively analyzing sites covalently-modified by the probe through DIA-based quantitative omics technique comprises: performing mass spectrometry analysis; acquiring data in DDA mode; generating a spectral library with the acquired data by Pulsar software; setting the number of windows and window intervals while reserving a 1.0 Da overlap between each two adjacent isolation windows; acquiring data in DIA mode from the sample with the same chromatography; and finally analyzing the DIA data results with Spectronaut.

In one embodiment, the method further comprises generating an electrophilic fragment molecule library, wherein the electrophilic fragment comprises acrylamide, chloroacetamide, ethylene oxide, acrylonitrile or ethyl vinyl sulfone as a reactive group.

In one embodiment, the electrophilic fragment molecule library is:

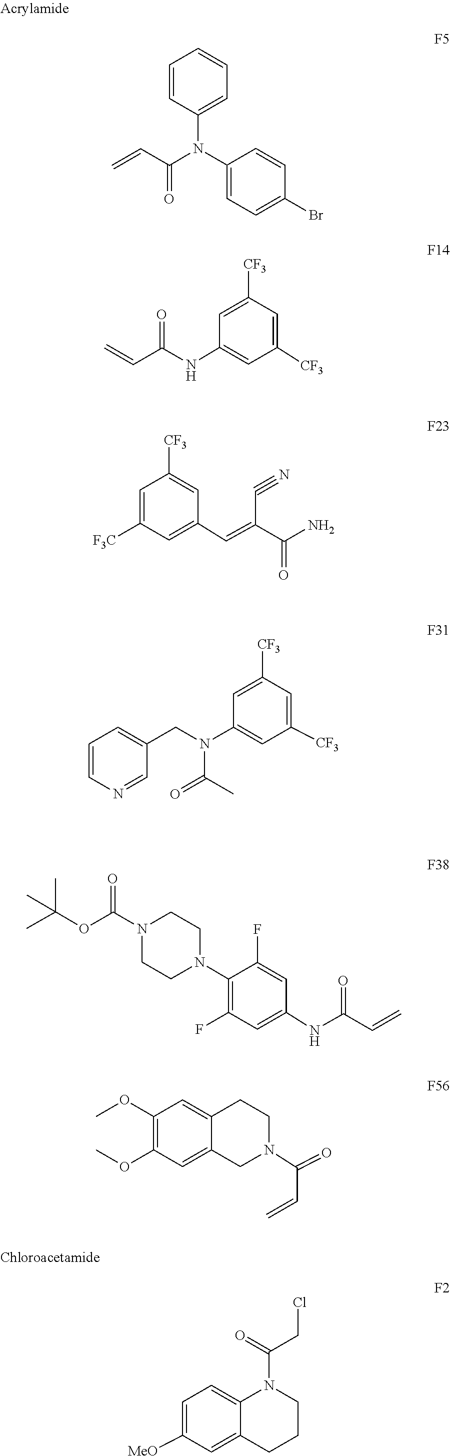

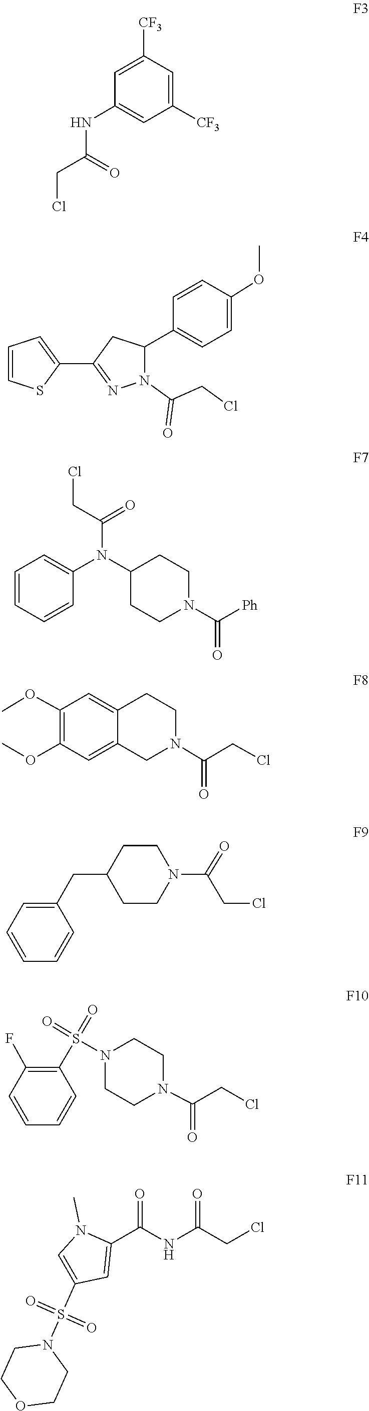

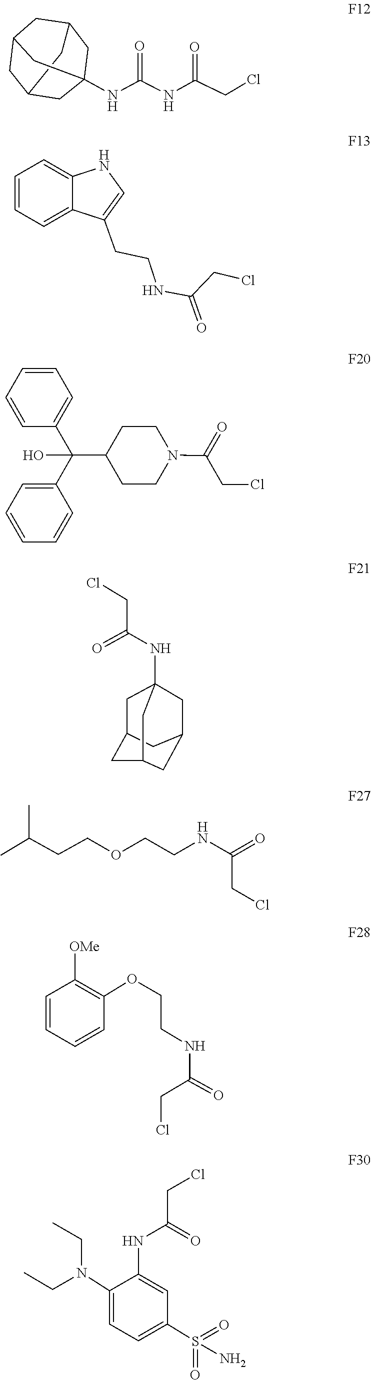

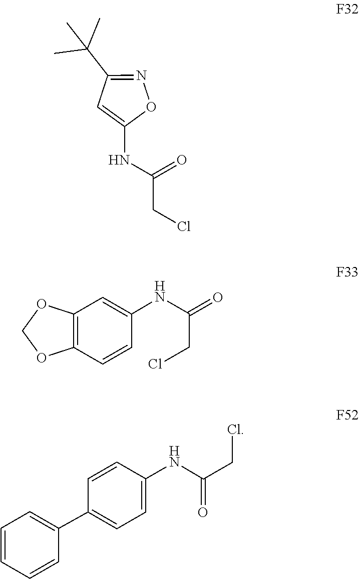 [SMILES: CC(C)(C)c1cc(NC(=O)CCl)on1 ; O=C(CCl)Nc1ccc2c(c1)OCO2 ; O=C(CCl)Nc1ccc(-c2ccccc2)cc1]

The present application constructs an electrophilic fragment molecule library, wherein the electrophilic fragments with acrylamide as a reactive group are numbered: F5, F14, F23, F31, F38, and F56 and the electrophilic fragments with chloroacetamide as a reactive group are numbered: F2, F3, F4, F7, F8, F9, F10, F11, F12, F13, F20, F21, F27, F28, F30, F32, F33, and F52. See FIG. 4 for detail.

In one embodiment, the method further comprises treating the proteome sample with the electrophilic fragment; then treating the sample with addition of the active molecular probe in the dark; then subjecting the treated sample to a click chemistry reaction with the cleavable tag reagent, enrichment and digestion and acid-cleavage; preparing a control sample by replacing the electrophilic fragment with dimethyl sulfoxide; preparing a sample labeled with the active molecular probe for DDA analysis to generate a spectral library; after setting windows, performing DIA acquisition and analysis on the sample having the electrophilic fragment.

In one embodiment, the sample comprises Ramos (human B lymphocytoma cells) cells, a tissue sample or a blood sample.

In one embodiment, the sample comprises human B lymphocytoma cells.

In one embodiment, the method further comprises treating the proteome sample with the electrophilic fragment; then treating the sample with addition of IA-alkyne probe in the dark; then subjecting the treated sample to a click chemistry reaction with the acid cleavable tag, enrichment and digestion and acid-cleavage; preparing a control sample by replacing the electrophilic fragment with dimethyl sulfoxide; preparing a human B lymphocytoma cell sample labeled with IA-alkyne probe for DDA analysis to generate a spectral library; after setting windows, performing DIA acquisition and analysis on the sample having the electrophilic fragment.

In one embodiment, the analysis of the sample having the electrophilic fragment comprises: for each active amino acid, the ratio of peptide segment intensity in the control sample to peptide segment intensity in the electrophilic fragment-treated sample is a targeting ratio of the electrophilic fragment binding to the active amino acid, and the median ratio of two replicates is reported as a final ratio. Active amino acids with final ratio less than 3 were screened and eliminated to obtain a final active amino acid quantitative information, and the active amino acid sites containing at least two fragments with a final ratio value greater than 4 and at least one fragment with a final ratio value between 0.5 and 2 were determined as targetable active amino acid sites.

In one embodiment, the analysis of the sample having the electrophilic fragment comprises: for each cysteine, the ratio of peptide segment intensity in the control sample to peptide segment intensity in the electrophilic fragment-treated sample is a targeting ratio of the electrophilic fragment binding to the cysteine, and the median ratio of two replicates is reported as a final ratio. Cysteines with final ratio less than 3 were screened and eliminated to obtain a final cysteine quantitative information, and the cysteine sites containing at least two fragments with a final ratio value greater than 4 and at least one fragment with a final ratio value between 0.5 and 2 were determined as targetable cysteine sites.

In one embodiment, the Pulsar parameters were set as follows: variable modification of cysteine carbamidomethylation+57.02146 Da, and variable modification introduced by IA-alkyne probe and acid cleavable tag on cysteine+280.18993 Da.

In the present application, DIA-ABPP, which combines DIA with ABPP, is a label-free quantitative technique. Theoretically, compared with labeled quantification, the relative quantitative error of DIA-ABPP is larger. The existing technique isoTOP-ABPP mainly relies on primary spectrum quantification. When acquiring samples, the acquisition time of the primary spectrum generally accounts for less than 20% of the total time, and the spectrum is highly complex and has many interferences. SLC-ABPP relies on secondary spectrum quantification. The interference of co-eluting peptides in secondary spectrum quantification will lead to the compression effect of quantitative ratio. However, the isobaric label-based multiple quantitative chemical proteomics technique (SLC-ABPP) developed by Gygi's team does not have this problem, because it isolates high-intensity fragment ions for tertiary mass spectrometry analysis. Nevertheless, it requires the latest generation of mass spectrometry and complex parameter settings, so its algorithm is complex and has poor operability.

Data acquisition mode-data-independent acquisition, can solve the data loss problem of conventional mass spectrometry acquisition mode—data-dependent acquisition (DDA). DIA technique can obtain sample information and accurate quantification without omission and difference under label-free conditions. The present application combines DIA technique with ABPP technique to form a new method called DIA-ABPP. This method avoids the use of expensive isotope tag reagents and serious data loss problems in conventional strategies. It is a new method with high coverage, high reproducibility and high precision. Since different types of reactive groups have different preferences for cysteine, the present application constructs an electrophilic fragment library containing chloroacetamide and acrylamide, and further uses this method to deeply explore targetable cysteine sites and discover regulatable proteins on a large scale, providing corresponding technical support for subsequent drug development.

DIA uses secondary spectrum quantification. The scanning time of the secondary spectrum generally accounts for about 90% of the total sample acquisition time, so a large amount of time allows to provide sufficient data quantitative information. In addition, the complexity of the mass spectrometry signal is reduced due to the isolation of the quadrupole. The present application also adds a retention time-correcting polypeptide at the chromatographic level, so that the retention time can be corrected, allowing for assignment to the correct peptide segment and the corresponding fragment ion. Combining the above related factors, from a quantitative point of view, DIA-ABPP is a quantitative chemical proteomics strategy with relatively high quantitative accuracy and precision, which is suitable for large-scale high-throughput detection and analysis.

In addition, the reference document (CN 112485442 A) discloses a chemical proteomics method for screening small molecule targets and discovering small molecule binding targets from complex biological sample systems, i.e., the target responsiveness accessibility profiling (TRAP), which uses active molecular probes to covalently modify specific active amino acids (such as lysine) in proteins in a biological sample (such as a cell lysate) containing target small molecule targets. After the labeling process, quantitative proteomics technique is used to compare the changes in the abundance of modified active amino acids at the whole proteome level in the biological sample in the absence and presence of incubated small molecules, thereby characterizing the accessibility changes of protein sites. Based on the multiplicity and significance of the changes, sites where chemical accessibility is significantly changed after incubation with the small molecule ligands are selected as candidate targets and potential binding and conformation-inducing sites of the small molecule ligands and targets.

The sample preparation method of the present application is different from that of the reference document (CN 112485442 A), and the proteomics methodology is also different (it is a combination of ABPP and DIA in the present application, which is different from the chemical proteomics used in the reference document). The subsequent screening process and screening criteria for specific active amino acids and target proteins are different, and the technical problems solved by the present application and the reference document are also different. The present application covalently modifies specific active amino acids in a proteome through an active probe molecule; then quantitatively analyze the sites covalently-modified by the probe through DIA-based quantitative omics, to obtain candidate targets. In addition, the screening coverage of the present application is wider, the reproducibility is good, and the accuracy is high.

### BRIEF DESCRIPTION OF THE DRAWINGS

In order to more clearly illustrate technical solutions of the present application, a brief introduction will be given below to the drawing necessarily used in the embodiments. Apparently, for those of ordinary skill in the art, other drawings can also be obtained based on these drawings without creative efforts.

FIG. 1 shows the comparison between DDA-based acquisition and DIA-based acquisition, where a is the comparison of the number of peptide segments identified by DDA sample and DIA sample acquisitions, and b is the comparison of the reproducibility of DDA sample and DIA sample acquisitions;

FIG. 2 shows the experimental result of the optimization of various parameters in the

DIA-ABPP technique, where a is the optimization of the cleavable tag, b is the optimization of the chromatography time, c is the peak corresponding to the same peptide segment in the 140 min chromatography and the 460 min chromatography, d is the optimization of the chromatography gradient, e is the optimization of the library search software, and f is the optimization of the spectral library acquisition;

FIG. 3 shows the evaluation of the identification and quantitation effect of DIA-ABPP technique, where a is the experimental process of DIA-ABPP technique, b is the comparison of DDA acquisition and DIA acquisition, c is the identification result of DIA-ABPP before and after optimization, d is the evaluation of DIA-ABPP technique under the same ratio condition, and e is the evaluation of DIA-ABPP technique under different ratio conditions;

FIG. 4 shows the molecule library of electrophilic fragments;

FIG. 5 shows the screening based on electrophilic fragments using DIA-ABPP technique, where a is the experimental process, b is the DIA-ABPP identification results and the functional analysis of targetable cysteine, c is the coverage of DIA-ABPP and isoTOP-ABPP technique, d is the number of identified cysteines corresponding to each protein, e is the cysteine targeting analysis on protein NUP205, f is the cysteine targeting analysis on protein NUP205, g and h are the reactivity analysis of fragments with different reactive groups of the same binding fragment.

### DETAIL DESCRIPTION OF THE INVENTION

The following embodiments are described in detail, and examples thereof are shown in the accompanying drawings. When the following description refers to the drawings, unless otherwise indicated, the same numbers in different drawings represent the same or similar elements. The embodiments described in the following example do not represent all embodiments consistent with the present application. They are only examples of systems and methods consistent with some aspects of the present application as detailed in the claims.

### EXAMPLE 1

### Cell Culture

K562 and Ramos cells were cultured with RPMI 1640 medium (Gibco, Life) and DMEM medium (Gibco, Life) at 37° C. and 5% CO2, respectively, which contain 10% fetal bovine serum (Thermo Fisher Scientific) and 1% penicillin-streptomycin (Thermo Fisher Scientific).

### EXAMPLE 2

### Establishment of DIA-ABPP Technique

The present application used the classic IA-alkyne probe labeled TOP-ABPP sample for establishment of a DIA mode-based method and subsequent condition optimization. The specific sample preparation could be found in the literature:

Weerapana Eranthie, Speers Anna E, Cravatt Benjamin F. Tandem orthogonal proteolysis-activity-based protein profiling (TOP-ABPP)—a general method for mapping sites of probe modification in proteomes. [J]. Nature protocols, 2007, 2 (6), and this technique is well known to those skilled in the art. 2 mg/mL K562 cell proteome samples were obtained for 1 mL each, treated with IA-alkyne at a final concentration of 100 μM at room temperature in the dark for 1 h, coupled with a TEV tag using a click chemistry reaction, and precipitated. After enrichment and trypsin digestion overnight, each sample was washed 3 times with 600 μL PBS and 3times with 600 μL deionized water to remove non-specifically adsorbed peptide segments and urea. After adding 500 μL TEV buffer to the streptavidin magnetic beads and washing once, 150 μL TEV buffer and 5 μL TEV enzyme were added, and digestion was carried out at 29° C. for 16 hours. Each sample was washed with 2× 50 μL water, the supernatant was collected and combined, and then spun dry, desalted, and the reagent for retention time correction (indexed retention time kit, iRT kit) was added and re-spun. Three samples were sent to the mass spectrometer for DDA mode acquisition. For subsequent analysis, the library was searched by ProLuCID with search parameters set to be: fixed modification of cysteine carbamidomethylation (+57.02146 Da), and variable modification introduced by IA-alkyne probe and TEV tag on cysteine (+464.28596 Da). The mass-to-charge ratio information corresponding to the parent ion of each corresponding modified peptide segment was obtained. The raw data was further analyzed to obtain the average peak width of the parent ion. According to: peak width=(primary spectrum scanning time+number of windows×secondary spectrum scanning time)×7, the number of windows can be finally calculated, and the interval of each window can be obtained according to the identification results. See Table 1 for the settings of mass spectrometry parameters.

TABLE 1
Mass spectrometry parameter settings
Mass spectrometry selective ion monitoring (Full MS-SIM)

| Resolution | 70000 | |
|---|---|---|
| Automatic Gain Control (AGC) | 3.00E+06 | |
| Maximum Injection Time | 20 | ms |
| (Maximum IT) | | |
| Scan range | 350 to 1800 | m/z |

| | |
|---|---|
| Polarity | Positive |

Data Independent Acquisition (DIA)

| Resolution | 17500 | |
|---|---|---|
| Automatic Gain Control (AGC) | 1.00E+06 | |
| Maximum Injection Time | Auto | |
| (Maximum IT) | | |
| Fixed first mass | 200 | m/z |
| Collision energy (N)CE/stepped | 28% | |
| nce | | |

| | |
|---|---|
| Default charge | 3+ |
| Microscans | 1 |
| Spectrum data type | Profile |

The results of DIA acquisition first underwent Pulsar library search and then were integrated into Spectronaut for comparison. The database used was Homo sapiens UniProt database (release-2012_11). The library search parameters were set as follows: fixed modification of cysteine carbamidomethylation (+57.02146 Da), variable modification introduced by IA-alkyne probe and TEV tag on cysteine (+521.30742 Da), protein false discovery rate (protein FDR)=1, peptide spectrum matching (PSM) false discovery rate=0.01 and peptide FDR=0.01, and iRT correction R²>0.8. After library search and the construction of the spectral library, the data acquired by three-needle DIA were further used for fragment ion matching, extraction and quantification, with a p value less than 0.01, and the final quantitative information was output according to the parent ion level.

### EXAMPLE 3

### Optimization of DIA-ABPP Technique

### (1) Cleavable Tag Reagent

According to the above experimental process, 2 mg/mL K562 proteome samples were obtained for 1 mL each, and treated with IA-alkyne probe with a final concentration of 100 μM at room temperature in the dark for 1 h. Subsequently, click chemistry reactions were carried out to install TEV tag, Diazo tag, Photo tag, and Acid tag, respectively. After enrichment and trypsin digestion overnight, non-specifically adsorbed peptide segments and urea were washed off with PBS and deionized water. Finally, cleavage was performed using the cleavage methods corresponding to different tag reagents.

### (2) Optimization of Chromatography Conditions

See Table 2 for the optimized chromatography time; see Table 3 for the optimized chromatography gradient.

TABLE 2
Optimized chromatography time

| | | |
|---|---|---|
| Chromatography | Chromatography | Chromatography |
| time 1/min | time 2/min | time 3/min |

| | | | | | |
|---|---|---|---|---|---|
| 0 | 7 | 0 | 7 | 0 | 7 |
| 40 | 20 | 70 | 20 | 125 | 20 |
| 120 | 30 | 235 | 30 | 385 | 30 |
| 140 | 80 | 280 | 80 | 460 | 80 |

TABLE 3
Optimized chromatography gradient

| Chromatography 1 | | Chromatography 2 | | Chromatography 3 | |
|---|---|---|---|---|---|
| 0 | 7 | 0 | 7 | 0 | 7 |
| 40 | 20 | 65 | 20 | 55 | 20 |
| 120 | 30 | 120 | 30 | 140 | 30 |
| 140 | 80 | 140 | 95 | 142 | 90 |

### (3) Selection of the Search Software Used for Library Generation

Three search software, ProLuCID, Thermo Proteome Discover, and Pulsar, were used to search the DDA data. All three used the same database: Homo sapiens UniProt database (release-2012_11). ProLuCID parameters were set to fixed modification of cysteine carbamidomethylation (+57.02146 Da) and variable modification introduced by IA-alkyne probe on cysteine (+464.28596 Da); Thermo Proteome Discover parameters were set to variable modification of cysteine carbamidomethylation (+57.02146 Da) and variable modification introduced by IA-alkyne probe on cysteine (+521.30742 Da); and Pulsar parameters were set to variable modification of cysteine carbamidomethylation (+57.02146 Da) and variable modification introduced by IA-alkyne probe on cysteine (+521.30742 Da). All three software finally obtained the false positive rate to 1%.

### (4) Optimization of Mass Spectrometry Conditions

### Isolation Window

The edge of the isolation window was accompanied by signal attenuation. It is necessary to reserve 1 Da overlap between each two adjacent isolation windows to alleviate the decrease in identification ability caused by the edge effect.

### (5) Construction of Spectral Library

The two prepared TOP-ABPP samples were pre-fractionated into 6 fractions for spectral library generation. The same two TOP-ABPP samples were directly subjected to mass spectrometry analysis without fractionation so as to generate the spectral library. According to the results of the two spectral libraries, the DIA method was set up for acquisition and subsequent Spectronaut analysis.

(6) The optimized Experimental Process is as Follows

2 mg/mL proteome sample were obtained for 1 mL each, treated with IA-alkyne probe at a final concentration of 100 μM at room temperature in the dark for 1 h, coupled with an acid tag using a click chemistry reaction, and precipitated. After enrichment and trypsin digestion overnight, non-specifically adsorbed peptide segments and urea were washed off. 200 μL 2% formic acid aqueous solution was added and reacted at 29° C. for 1 h, repeated. It was washed twice with 1% formic acid and 50% acetonitrile aqueous solution. The supernatant was collected and combined, spin-dried, desalted, added with a reagent for retention time correction (indexed retention time kit, iRT kit), re-spun, and sent to the mass spectrometer for DDA mode acquisition with the chromatography conditions of 140 min gradient 1. The obtained data results were used to generate the spectral library by Pulsar, the number of windows and window intervals were set, and data was acquired in DIA mode from the sample with the same chromatography; and finally, the DIA data results was analyzed with Spectronaut.

It should be noted that the room temperature involved in the example of the present application was 29° C.

### EXAMPLE 4

### Quantitation Evaluation of DIA-ABPP Technique

The sample preparation was completed according to the above process to obtain the TOP-ABPP sample, which was dissolved in triplicate by adding 0.1% formic acid aqueous solution containing iRT kit, and was used for DDA mode library generation. The inclusion list and isolation windows and other mass spectrometry means were set. 4 samples were prepared subsequently in a content ratio of 1:2:5:10 (with the same iRT content), and subsequent acquisition was performed in the DIA mode. Spectronaut was used for subsequent matching, extraction and quantification.

### EXAMPLE 5

### DIA-ABPP Technique for Screening Based on Electrophilic Fragments

2 mg/mL Ramos cell proteome samples were obtained for 1 mL each, treated with electrophilic fragments at a final concentration of 500 μM for 1 h, then treated with an IA-alkyne probe added at a final concentration of 100 μM at room temperature in the dark for 1 h, and subjected to a click chemistry reaction to install an acid tag. Subsequent enrichment, digestion and acid-cleavage were performed. Control samples were prepared in which the electrophilic fragments were replaced with DMSO (dimethyl sulfoxide). 2 samples were prepared for each electrophilic fragment, and 6 control samples were prepared. Six IA-alkyne probe-labeled samples of Ramos cells were further prepared for DDA analysis to generate a spectral library. After completing the window setting, DIA acquisition and analysis of 6 control samples and 48 samples for electrophilic fragments were performed. For each cysteine, the ratio of the peptide segment intensity in the control sample to the peptide segment intensity in the electrophilic fragment-treated sample was the targeting ratio of the electrophilic fragment binding to the cysteine, and the median ratio of the two replicates was reported as the final ratio (R). Cysteine with values less than 3R were screened and eliminated to obtain a final cysteine quantitative information list.

Further, the cysteine sites containing at least two fragments with a R value greater than 4 and at least one fragment with a R value between 0.5 and 2 were determined as targetable cysteine sites.

The following comparative experiments will illustrate the beneficial effects of the method for DIA-based screening targets by quantitative chemical proteomics provided in the present application.

### EXAMPLE 6

### Comparison of Identification Results Obtained After Acquiring Mass Spectrometry Data in DDA and DIA Modes

See FIG. 1, which shows a comparison between DDA acquisition and DIA acquisition.

Six K562 cell proteome samples were labeled with 100 μM IA-alkyne probe. 6 samples with IA-alkyne probe modification site on the cysteine of the peptide segment were obtained through the TOP-ABPP experimental process, and the acquisition was performed in DDA and DIA modes with spectral library construction based on DDA. FIG. 1 summarizes the identification results obtained after acquiring mass spectrometry data from three samples in DDA and DIA modes, respectively. a is the number of peptides identified in three DIA samples and three DDA samples; b is the number of peptide segments repeatedly identified in three DIA samples and three DDA samples. The horizontal axis is the experimental number of the three samples, and the vertical axis is the intersection of the number of IA-alkyne probe-modified peptide segments identified in the sample and the number of modified peptide segments identified in the previous one or two samples, which is used to reflect the overall level and reproducibility of the method for identification of modified peptides.

When only three samples were analyzed, the types of peptide segments identified in all three samples by the DDA method were only about half of those in a single sample. The results of DIA were in sharp contrast to those of DDA. Since all parent ions were assigned to different charge-to-mass ratio windows for acquisition and fragmentation in the DIA mode, the information of modified peptide segments was retained as much as possible, and the number of peptide segments identified in all three samples could be maintained at more than 90% of that in a single sample. The above results showed that the reproducibility of modified peptide segment identification in chemical proteomics data could be greatly improved by DIA acquisition mode.

### EXAMPLE 7

### Effect of Optimizing Experimental Parameters

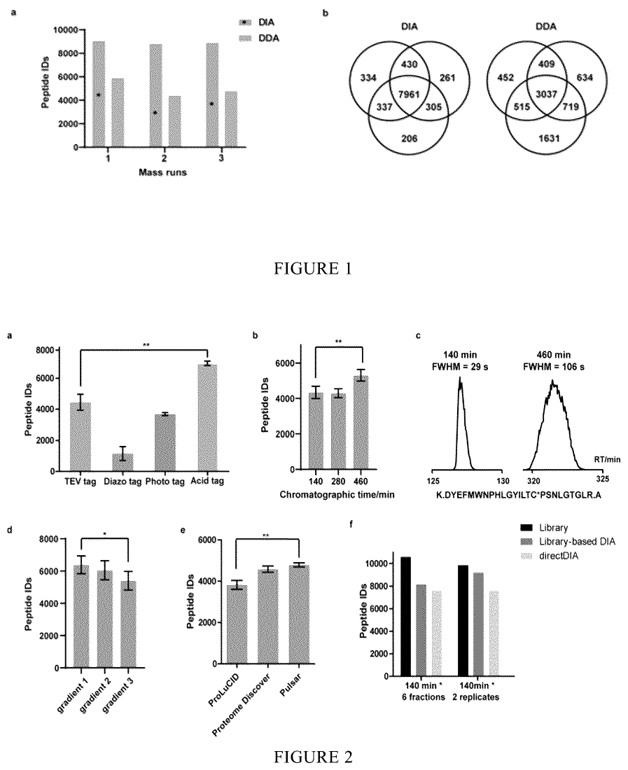

See FIG. 2, which shows the experimental result of the optimization of various parameters in the DIA-ABPP technique.

The DIA-ABPP process was systematically optimized through sample preparation, chromatography condition, library search software, mass spectrometry condition, and spectral library acquisition.

FIG. 2 shows the experimental results after parameter optimization in each aspect. When mass spectrometry analysis was performed under the conditions of acid tag, 140 min, and high acetonitrile phase chromatographic gradient and the spectral library was generated using the library search software Pulsar, DIA data gave the best result. In addition, in the quadrupole, the edge of the isolation window was accompanied by signal attenuation. It is necessary to reserve 1.0 Da overlap between each adjacent isolation windows in the present application to compensate the decrease in identification ability caused by the edge effect.

In FIG. 2, a The effects of four cleavable tag reagents commonly used in chemical proteome analysis on identification were compared using TOP-ABPP. Acid-cleavage tag has the best performance as it facilitates sample preparation, and is conducive to mass spectrometry detection; b the number of peptides identified corresponding to three liquid chromatography times was compared using TOP-ABPP; c the chromatographic peak width of the same peptide segment under 140 min and 460 min chromatography times was analyzed; although longer elution times allowed for identification of more peptides, the chromatographic peak of the 460 min spectrum was significantly widened, which would interfere with downstream DIA analysis; d the number of peptides identified by TOP-ABPP under three chromatography gradients was compared. The higher the organic phase ratio, the better the gradient effect; e the number of peptide segments identified by three search engines (including ProLuCID, Proteome Discoverer and Pulsar) was compared. Pulsar generated the largest spectral library; f the number of peptides identified using a pre-acquired spectral library (the sample is pre-fractionated to generate a spectral library) and directDIA (without pre-acquiring a spectral library) was prepared. Although the spectral library generated without pre-fractionation was slightly smaller than the spectral library generated with fractionation, which may be due to the loss of low-intensity peptides, the former has the best DIA identification effect (values represent the mean±SD of three repeated experiments. *p<0.05, **p<0.01, t test).

### EXAMPLE 8

### Identification and Quantitation Evaluation of DIA-ABPP Technique

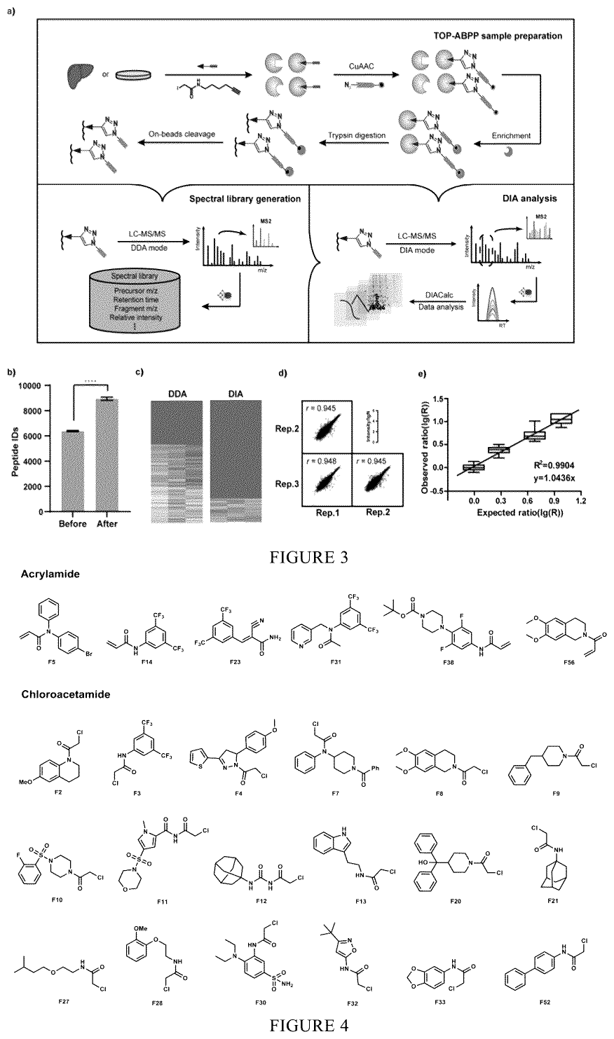

See FIG. 3, which is the identification and quantitation evaluation of DIA-ABPP technique.

After the completion of optimizing all the above conditions, the present application completed the acquisition of three shots of DDA samples which guided the acquisition and mass spectrometry data analysis of the subsequent another three shots of DIA samples after library construction (FIG. 3a). As can be seen in the comparison in FIG. 3b, the optimized DIA-ABPP technique can identify 50% more modification sites than DDA-ABPP. The data loss of samples acquired by DDA was very serious, while the data preservation during DIA acquisition was complete, with better coverage and reproducibility.

The present application prepared TOP-ABPP samples modified by IA-alkyne probes for quantitative effect evaluation. First, three identical samples were analyzed using DIA-ABPP technique, and it was found that there was a good consistency between the samples (FIG. 3d). In order to further compare the accuracy and precision of quantification between samples of different contents, the applicant prepared seven identical samples, three of which completed the data acquisition in DDA mode for the generation of a spectral library, and then the other four samples were divided into four samples according to the content ratio of 1:2:5:10. The mass spectrometry data was acquired, compared, extracted and subsequently quantified using the DIA mode. FIG. 3e is the quantitative experimental result, in which the horizontal and vertical axes represent the theoretical ratio and the experimental measurement ratio, respectively. The present application extracted the median of the theoretical ratio and the measurement ratio for linear fitting through zero point. The box plot results show that the ratio of peptide quantification is relatively concentrated and has high accuracy. The result of linear fitting is y=1.0436×, and R² is 0.9904, indicating that the new method has high accuracy.

FIG. 3 is the evaluation of the identification and quantitation of DIA-ABPP technique. Among them are: a DIA-ABPP solution, including TOP-ABPP sample preparation, spectral library generation and DIA analysis (optimization conditions: using an acid-cleaved tag for labeled peptide release, using high acetonitrile gradient, 140 min chromatography time, and using Spectronaut to process DIA data based on the spectral library generated by Pulsar); b the number of peptide segments identified by TOP-ABPP experiments before and after optimization; c the number of peptides identified by samples acquired by DDA and DIA; d correlation analysis between DIA-ABPP samples: the Pearson correlation (r) between samples is calculated; e quantitation performance analysis of DIA-ABPP: the DIA analysis of samples at a prescribed ratio of 1:2:5:10, plotting the results of the ratio measured by DIA-ABPP. The box plot shows the median, 10th percentile and 90th percentile.

### EXAMPLE 9

### Screening of Electrophilic Fragments Using DIA-ABPP Technique

See FIG. 4, which is the molecule library of electrophilic fragments.

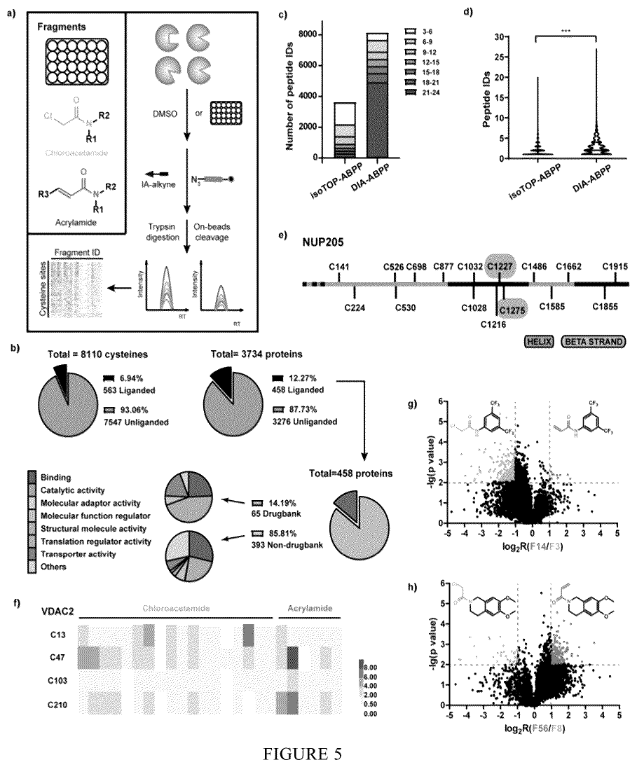

See FIG. 5, which is the screening based on electrophilic fragments using DIA-ABPP technique.

Given the high reproducibility and high coverage of DIA-ABPP technique, the applicant established a molecule library of 24 electrophilic fragments (FIG. 4), and further used the proteome of Ramos cells to quantitatively analyze the level of electrophilic fragments binding to cysteine (FIG. 5a). In a total of 54 DIA samples, the applicant identified 8110 cysteine sites in 3734 proteins, and the instrument time used was one-fourth of that of the prior method (isoTOP-ABPP). It is worth noting that 67.35% of the cysteine in the DIA-ABPP identification results had quantitative data for at least 21 fragments, while the coverage of isoTOP-ABPP technique was only 5.8% (FIG. 5c).

FIG. 4 shows the chemical structures of the 24 electrophilic fragments used in this study. These fragments contain acrylamide or chloroacetamide as a reactive group. FIG. 5 shows the screening based on electrophilic fragments using DIA-ABPP. a schematic diagram of the reactive groups of covalent fragments and the application process for screening using DIA-ABPP; b the proportion of targetable cysteines and proteins obtained by DIA-ABPP strategy analysis. The corresponding proteins were further searched using the DrugBank database and classified according to the corresponding functional categories; c comparison of the number distribution of cysteines quantified by electrophilic fragments when analyzed using isoTOP-ABPP and DIA-ABPP techniques (cysteines with quantitative results for at least three fragments are listed); d violin plots showing the distribution of the number of cysteines quantified for each protein when analyzed by isoTOP-ABPP and DIA-ABPP techniques. (***p<0.001, t-test); e analysis of targetability of cysteine in protein NUP205 by the DIA-ABPP technique, two targetable cysteine sites being showed with color depths of 8.0 and 6.0; f heat map showing the targetability of four cysteine sites in VDAC2 to 24 electrophilic fragments; g-h. comparison of the differences in the targetability of electrophilic fragments having the same binding fragments but different reactive groups to cysteine in volcano plots.

It should be noted that the applicant uses the same standard for defining “linkable cysteine” in the prior article (Backus Keriann M, Correia Bruno E, Lum Kenneth M. Proteome-wide covalent ligand discovery in native biological systems. [J]. Nature, 2016, 534 (7608)) (for example, the level of cysteine competitive probe is higher than 75% by labeling with two or more ligand fragments), and identified 563 targetable cysteines from the DIA-ABPP data set, which has a similar targetability level as the prior art. These targetable cysteines belong to 458 proteins, 85% of which do not appear in the drug library database and are involved in a variety of molecular functions such as binding, catalysis, regulation, and transport (FIG. 5b).

In addition, it is worth noting that for a single protein, the DIA-ABPP technique can obtain more quantitative information on cysteines (FIG. 5d), so that the position selectively targeted by the electrophilic fragment can be better determined. The most notable example is NUP205, which is an inner ring nucleoporin that plays a role in the assembly and/or maintenance of the nuclear pore complex (NPC). DIA-ABPP was able to quantify 16 cysteines in this protein, two of which are considered to be cysteine sites that can be targeted by different types of electrophilic probes; while for isoTOP-ABPP, only information on one inactive cysteine (C1662) was obtained (FIG. 5e). At the same time, the reactivity of different electrophilic reactive groups was analyzed, and molecules having the same binding fragment but different reactive groups in the molecule library were selected for head-to-head comparison. The applicant found that they showed different reactivity to cysteine, indicating that different reactive groups have different preferences for cysteine (FIG. 5g, 5h). When further protein-centered analysis was conducted, it was found that four cysteines of protein VDAC2 were identified during analysis by DIA-ABPP technique, and they had different linking abilities to molecules containing different reactive groups. Among them, C210, one of the cysteines that were hypersensitive to the lipid-derived electrophilic small molecule HNE, could only be targeted by fragments containing acrylamide, and such translation group was similar to the reactive group in HNE (FIG. 5f).

The similar parts among the examples provided in the present application can be referred to each other. The specific implementations provided above are only a few examples under the general concept of the present application and do not constitute a limitation on the scope of protection of the present application. For those skilled in the art, any other implementations expanded based on the solution of the present application without creative work belongs to the scope of protection of the present application.

What is claimed is:

1. A method for screening targets by DIA-based quantitative chemical proteomics, comprising:
covalently modifying specific active amino acids in a proteome with a reactive molecular probe; and
quantitatively analyzing sites covalently-modified by the probe through DIA-based quantitative omics technique, to obtain candidate targets.

2. The method for screening targets by DIA-based quantitative chemical proteomics of claim 1, wherein the active amino acids are cysteine, lysine, tyrosine, methionine, glutamic acid or aspartic acid.

3. The method for screening targets by DIA-based quantitative chemical proteomics of claim 2, wherein the correspondence between the active amino acid and the active molecular probe is: the cysteine being covalently-modified by an IA-alkyne probe, the lysine being covalently-modified by a tetrafluorophenylsulfonate-alkyne probe, the tyrosine being covalently-modified by a sulfonyltriazole-substituted alkyne probe, the methionine being covalently-modified by an oxazine-alkyne probe, and the glutamic acid and aspartic acid being covalently-modified by a 3-phenyl-2H-aziridine-alkyne probe.

4. The method for screening targets by DIA-based quantitative chemical proteomics of claim 3, wherein the correspondence between the active amino acid and the active molecular probe is: cysteine being covalently-modified by an IA-alkyne probe.

5. The method for screening targets by DIA-based quantitative chemical proteomics of claim 1, wherein, the process of covalently modifying specific active amino acids in a proteome with an active molecular probe, wherein a cleavable tag is used, specifically comprises:
obtaining a proteome sample; treating the sample with the active molecular probe in the dark; subjecting the treated sample to a click chemistry reaction with the cleavable tag;
after enrichment and digestion overnight, washing off non-specifically adsorbed peptide segments and urea, and finally performing cleavage using a cleavage method corresponding to the cleavable tag reagent.

6. The method for screening targets by DIA-based quantitative chemical proteomics of claim 1, wherein the process of quantitatively analyzing sites covalently-modified by the probe through DIA-based quantitative omics technique comprises: performing mass spectrometry analysis; acquiring data in DDA mode; generating a spectral library with the acquired data by a Pulsar software; setting the number of windows and window intervals while reserving a 1.0 Da overlap between each two adjacent isolation windows; acquiring data in DIA mode from the sample with the same chromatography; and finally analyzing the DIA data results with Spectronaut.

7. The method for screening targets by DIA-based quantitative chemical proteomics of claim 1, further comprising generating an electrophilic fragment molecule library, wherein the electrophilic fragment comprises acrylamide, chloroacetamide, ethylene oxide, acrylonitrile or ethyl vinyl sulfone as a reactive group.

8. The method for screening targets by DIA-based quantitative chemical proteomics of claim 7, wherein the electrophilic fragment molecule library is:

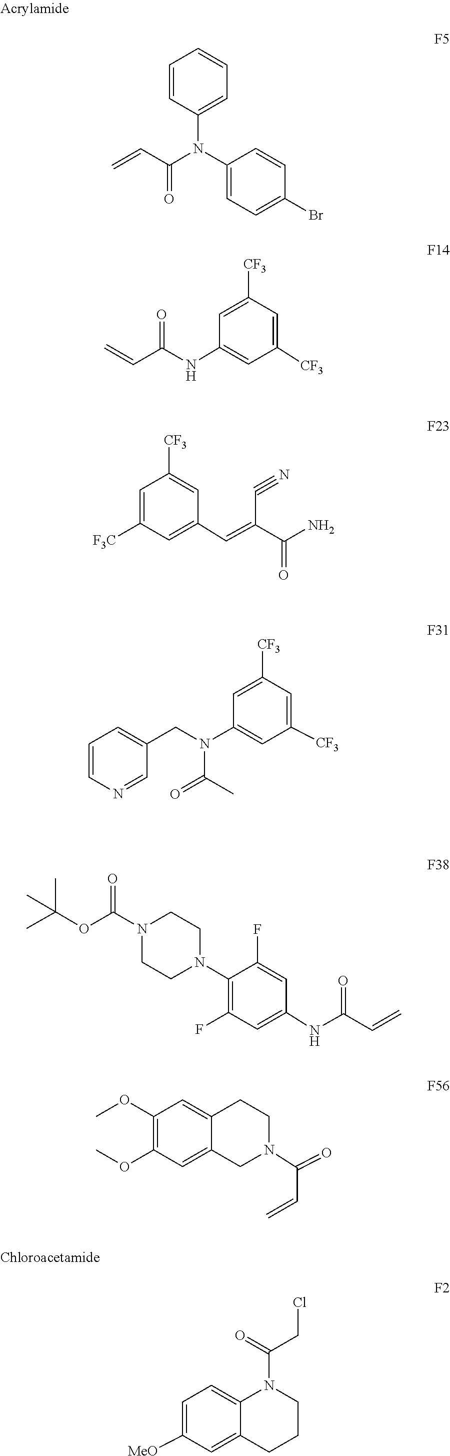

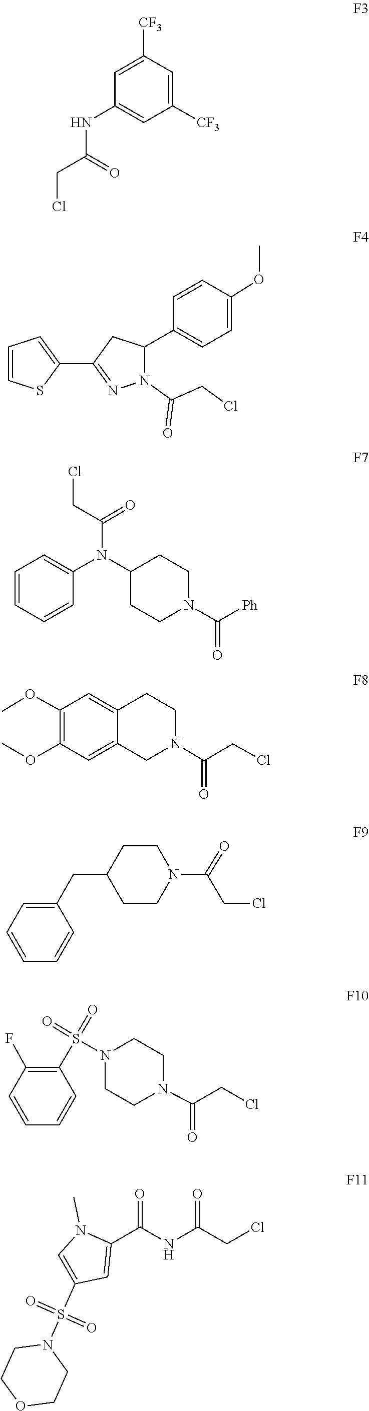

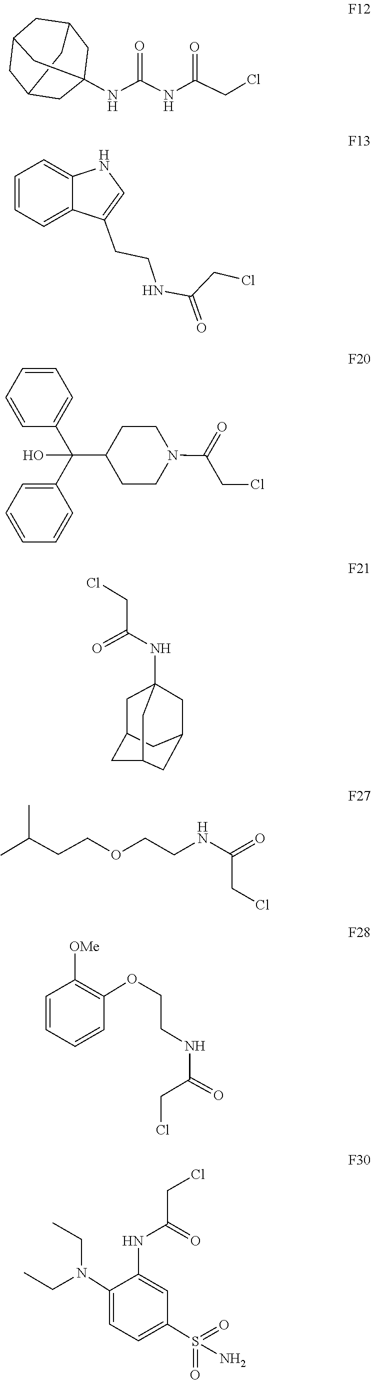

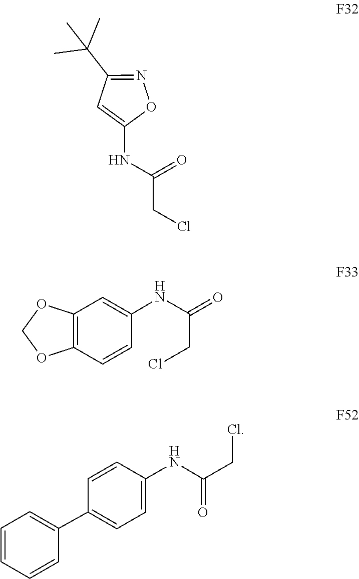 [SMILES: CC(C)(C)c1cc(NC(=O)CCl)on1 ; O=C(CCl)Nc1ccc2c(c1)OCO2 ; O=C(CCl)Nc1ccc(-c2ccccc2)cc1]

9. The method for screening targets by DIA-based quantitative chemical proteomics of claim 7, further comprising treating the proteome sample with the electrophilic fragment; then treating the sample with reactive molecular probe in the dark; then subjecting the treated sample to a click chemistry reaction with the cleavable tag reagent, enrichment and digestion and acid-cleavage; preparing a control sample by replacing the electrophilic fragment with dimethyl sulfoxide; preparing a sample labeled with the active molecular probe for DDA analysis to generate a spectral library; after setting windows, performing DIA acquisition and target analysis on the sample having the electrophilic fragment.

10. The method for screening targets by DIA-based quantitative chemical proteomics of claim 9, wherein the sample comprises human B lymphocytoma cells, a tissue sample or a blood sample.

11. The method for screening targets by DIA-based quantitative chemical proteomics of claim 9, wherein the analysis of the sample having the electrophilic fragment comprises: for each active amino acid, the ratio of peptide segment intensity in the control sample to peptide segment intensity in the electrophilic fragment-treated sample is a targeting ratio of the electrophilic fragment binding to the active amino acid, and the median ratio of two replicates is reported as a final ratio, and the active amino acid with less than 3 final ratio values is screened and eliminated to obtain a final active amino acid quantitative information, and the active amino acid sites containing at least two fragments with a final ratio value greater than 4 and at least one fragment with a final ratio value between 0.5 and 2 are screened, i.e., targetable active amino acid sites.
